(19) United States
(12) Patent Application Publication
(10) Pub. No.: US 2022/0218832 A1
(43) Pub. Date: Jul. 14, 2022

(54) POLYMER DRUG

(71) Applicant: BIODYNAMIC RESEARCH FOUNDATION, Kumamoto (JP)

(72) Inventors: Hiroshi MAEDA, Kumamoto-shi, Kumamoto (JP); Waliul ISLAM, Kumamoto-shi, Kumamoto (JP)

(73) Assignee: BIODYNAMIC RESEARCH FOUNDATION, Kumamoto (JP)

(21) Appl. No.: 17/442,818

(22) PCT Filed: Mar. 27, 2020
(86) PCT No.: PCT/JP2020/014322

§ 371 Date: Sep. 24, 2021

(30) Foreign Application Priority Data
Mar. 28, 2019 (JP) 2019-064562

(51) Int. Cl. A61K 47/58; C08F 8/42; A61K 41/00; A61P 35/00; A61P 31/04; A61K 33/22; A61K 31/7008

(52) U.S. Cl. CPC A61K 47/58; C08F 8/42; A61K 41/0095; A61K 31/7008; A61P 31/04; A61K 33/22; A61P 35/00

(57) ABSTRACT

The present invention provides a novel macromolecular drug, more specifically, a complex comprising a styrene-maleic acid copolymer (SMA) and a boric acid compound, wherein the SMA is bound to the boric acid compound directly or through a linker.

### TECHNICAL FIELD

The present invention relates to a macromolecular drug. The present application is filed claiming the priority of the Japanese Patent Application No. 2019-64562, the entire contents of which are herein incorporated by reference.

### BACKGROUND ART

The present inventors have discovered and reported the EPR effect (enhanced permeability and retention effect), which is the principle of selective accumulation of macromolecular compounds or materials in tumor sites (Non-Patent Literature 1: Cancer Research, 1986 (12), 46, 6787-6392). By applying the principle, the present inventors have found that macromolecular drugs comprising a low molecular weight drug linked to a biocompatible polymer can show the EPR effect and thus accumulate predominantly in tumor sites (Patent Literature 1: WO 2004/103409, Patent Literature 2: WO 2006/112361, etc.).

Thus, the binding of low molecular weight drugs with polymers may enhance the drug efficacy of low molecular weight drugs.

For example, boric acid has been conventionally used as a low molecular weight drug in antibacterial agents, bactericidal agents, insecticides, and pharmaceuticals. For example, so-called boric acid dumplings (10%-50%) are used as food poisoning agents for cockroach extermination, and boric acid solutions may be used for ant extermination. In the field of ophthalmology, boric acid is used to clean and disinfect conjunctival sacs or as a preservative in eye drops. Also, boric acid can be used as a neutralizer when basic chemicals get into the eyes. In the United States and Europe, boric acid is often applied to building wood as an insecticide and preservative against termites and fungi.

However, there is no known macromolecular agent comprising boric acid linked to a polymer.

On the other hand, in addition to surgery, cancer treatments include chemotherapy, photodynamic therapy (PDT), and radiation therapy, and more recently immunotherapy. Among cancer treatments, particularly radiation therapy, boron neutron capture therapy (BNCT) comprises administrating a medicament containing boron (¹⁰B) to a patient and irradiating the tumor sites with neutrons (thermal neutrons) generated by accelerators or by nuclear reactors. In this therapy, a ray generated upon this irradiation is considered to be the main cell killing factor.

However, all drugs or agents used in the above methods, classical or conventional chemotherapeutic agents, or photosensitizers used for PDT and borono-compounds for BNCT are generally low molecular weight drugs. Low molecular weight drugs freely and widely diffuse and distribute throughout the body and do not selectively accumulate in tumor sites, thereby resulting in poor therapeutic effects. In fact, both WHO (United Nations Health Organization) and the National Cancer Institute (NCI) in the United States have stated that 90±5% of the cancer treatments are failure (Non-patent Literature 2: Clin. Trans. Med. (2018) 7: 11/10.1186/s40169-018-0185-6).

### CITATION LIST

### Patent Literature

### [Patent Literature 1] WO 2004/103409

### [Patent Literature 2] WO 2006/112361

### Non-Patent Literature

### [Non-Patent Literature 1] Cancer Research, 1986 (12), 46, 6787-6392

[Non-patent Literature 2] Clin. Trans. Med. (2018) 7: 11

### SUMMARY OF INVENTION

### Technical Problem

An object of the present invention is to provide a novel macromolecular drug which is useful as, for example, an anticancer drug (particularly an anticancer drug for BNCT), an antibacterial agent, a bactericidal agent or the like.

### Solution to Problem

The present inventors have conducted intensive studies to achieve the above object and finally succeeded in making boric acid macromolecules by using a boric acid compound and a polymer. The boric acid macromolecules preferentially accumulate in the tumor sites than the normal tissues and organs by the EPR effect. Making such macromolecular conjugate can greatly improve therapeutic effects (anticancer effects) while reducing side effects. Furthermore, the present inventors have found that glucosamine conjugated with a macromolecule exhibits sufficient anticancer effects.

Furthermore, the present inventors have found that the above macromolecular boric acid compounds exhibit excellent antibacterial activity against both Gram-positive and Gram-negative bacteria.

On the basis of these findings, the present inventors have completed the present invention.

The present invention includes the followings:

[1] A complex comprising a styrene-maleic acid copolymer (SMA) and a boric acid compound, wherein the SMA is bound to the boric acid compound directly or through a linker.
[2] The complex according to [1], wherein the boric acid compound is selected from boric acid, disodium tetraborate, and mixtures thereof.
[3] The complex according to [1] or [2], wherein the linker is bound to the SMA via an amide bond, an ester bond, a thioester bond, or a hydrazone bond.
[4] The complex according to any one of [1] to [3], wherein the linker is selected from saccharides, amino sugars, sugar alcohols, and mixtures thereof.
[5] The complex according to any one of [1] to [3], wherein the linker is a cis-diol compound.
[6] The complex according to [1] or [2], wherein the SMA is directly bound to the boric acid compound.
[7] An anticancer agent comprising the complex according to any one of [1] to [6].
[8] The anticancer agent according to [7] for use in boron neutron capture therapy.
[9] A method for producing the complex according to any one of claims 1 to 5, which comprises the following steps:
(a) binding the linker to the SMA, and
(b) binding the linker residue of the product obtained in step (a) to the boric acid compound.
[10] An anticancer agent comprising a conjugate of styrene-maleic acid copolymer (SMA) and glucosamine.
[11] An antibacterial agent comprising the complex according to any one of [1] to [6].

### Effects of Invention

According to the SMA-boric acid complex of the present invention, it is possible to incorporate boron in a macromolecule by using a boric acid compound and a polymer, thereby allowing a larger amount of boron to be accumulated at tumor sites than other normal sites due to the EPR effect. As a result, the therapeutic effect (anticancer effect), particularly the therapeutic effect by BNCT can be significantly improved while the side effects can be reduced. Therefore, the complex of the present invention is far superior to a conventional low molecular weight anticancer drug or low molecular weight boron formulation for BNCT.

Also, the complex of the present invention can liberate the boric acid compound at tumor sites, and the free boric acid compound can inhibit the metabolism of glycolytic system (Warburg effect), on which many cancer cells depend among metabolism systems for energy (ATP) generation in cells, and thus can suppress the growth of cancer cells.

Therefore, the complex of the present invention can exert anticancer effects in two mechanisms of glycolytic inhibition as well as therapeutic effect by BNCT.

The complex of the present invention can also inhibit glucose uptake into cells. Therefore, the complex can be used as a glucose uptake inhibitor, and can be used for diseases where this can improve symptoms.

Furthermore, according to the SMA-glucosamine conjugate of the present invention, it is possible to make glucosamine to be a macromolecule, thereby allowing a larger amount of glucosamine to be accumulated at the tumor sites than at other sites due to the EPR effect. As a result, the conjugate is slowly cleaved by hydrolases/proteases/amidases in tumor cells to liberate glucosamine. The free glucosamine can also exert anticancer effects. This is the third anticancer mechanism related to the present invention.

Since the complex of the present invention exhibits excellent antibacterial activity against both Gram-positive and Gram-negative bacteria, it can be used as antibacterial agents and can be used as a treatment or preventive agent for infections caused by these bacteria.

### BRIEF DESCRIPTION OF THE DRAWINGS

FIG. 1 shows infrared absorption spectrums of SMA, SMA-glucosamine conjugate (SG), and SMA-glucosamine-boric acid complex (SGB). In the figure, a peak indicated by an arrow corresponds to the amide bond.

FIG. 2A shows a UV spectrum of SMA. BSA refers to bovine serum albumin for comparison.

FIG. 2B shows a column chromatography with Sephacryl S-300 column (2 cm×60 cm, GE Healthcare). Transferrin (90 KDa), BSA (67 KDa) and neocarzinostatin (NCS) (12 KDa) were used as molecular weight standards. The apparent molecular weight of SGB +BSA changed from about 70 KDa to about 150 KDa. The molecular weight of the original SGB was about 65 KDa. This indicates that SGB has an albumin-binding property in a solution.

FIG. 3 shows an electron micrograph of SGB.

FIG. 4 shows a release curve of boric acid from SGB.

FIG. 5 shows a cytostatic effect by SGB, which was performed in vitro by using HeLa cells (1×10⁴/well) in a culture medium with 0.1% glucose under normal partial pressure of oxygen (O₂, 21%). The data show the cell viability values measured by MTT method 24 hours after drug treatment.

FIG. 6(A) and (A′) show a cytostatic activity of free glucosamine against C26 cells, mouse colon cancer cells, FIG. 6(B) and (B′) show a cytostatic activity of SMA-glucosamine conjugate (SG) against C26 cancer cells, and FIG. 6(C) shows a cytostatic activity of SG-boric acid complex (SGB) against C26 cancer cells. The (A), (B) and (C) show the results where C26 cells were cultured in an ordinary medium containing 0.1% glucose, and the (A′) and (B′) show the results where the cells were cultured in a medium containing a low concentration (0.01%) of glucose at a low pH and a low oxygen partial pressure. These conditions simulate the microenvironment of cancer tissue in highly advanced cancer.

FIG. 7A shows the in vitro cytotoxicity of SGB compared to free boric acid (BA) in C26 cells under normoxic and hypoxic partial pressures for 48 hours.

FIG. 7B shows the in vitro cytotoxicity of SGB compared to free boric acid (BA) in HeLa cells under normoxic and hypoxic partial pressures for 48 hours.

FIG. 7C shows the in vitro cytotoxicity of glucosamine and SMA-glucosamine in C26 cells under normoxic and hypoxic partial pressures for 48 hours.

FIG. 7D shows the in vitro cytotoxicity of glucosamine and SMA-glucosamine in HeLa cells under normoxic and hypoxic partial pressures for 48 hours.

FIG. 8 shows toxicity evaluation for a single intravenous injection of SGB, which was carried out by using a 6-week-old male ddY mice.

FIG. 9 shows distribution of SGB and free boric acid in organs and tumor tissues in tumor-bearing (S180) mice. The B10 (¹⁰B) (unit: ppb) was detected by ICP mass spectrometry at 24 hours after administration.

FIG. 10A shows the plasma half-lives of boric acid and SMA-glucosamine-boric acid complex in ddY mice.

FIG. 10B shows the urinary excretion rates of boric acid and SMA-glucosamine-boric acid complex in ddY mice.

FIG. 11 shows a comparison of the cell uptake of free boric acid and SGB in C26 cells.

FIG. 12A shows the inhibition of glucose uptake by SGB.

FIG. 12B shows lactate production by SGB.

FIG. 13A shows the antibacterial activity of SMA-glucosamine-boric acid complex against Staphylococcus aureus.

FIG. 13B shows the antibacterial activity of SMA-glucosamine-boric acid complex against Escherichia coli (E. coli).

### DESCRIPTION OF EMBODIMENTS

The present invention relates to a complex comprising a styrene-maleic acid copolymer (SMA) and a boric acid compound, wherein the SMA is bound to the boric acid compound directly or through a linker.

In the present invention, “styrene-maleic acid copolymer (SMA)” is a copolymer comprising a repeating unit represented by the following formula (1), which comprises a structural unit derived from styrene and a structural unit derived from maleic acid (including maleic anhydride) as essential component units. The SMA may be obtained in the market, or synthesized by known synthesis procedure. It is generally obtained by copolymerization of styrene and maleic anhydride. In this case, the residue from maleic anhydride in SMA will be an anhydride, which can be used as is, or as free carboxyl form after hydrolysis in alkaline before use.

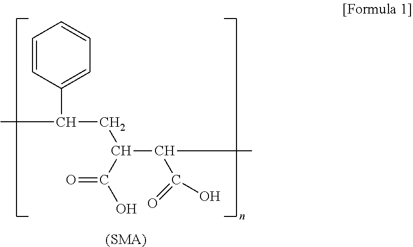

In the formula (1), n represents an integer of 2 or more, for example, 3 to 500.

In the present invention, the SMA may be a derivative thereof, in which various functional groups are introduced into the side chain portion of the maleic acid residue. Examples of the SMA derivative include those having a maleic acid side chain to which albumin or transferrin is conjugated; those having a maleic acid side chain of which the carboxyl group is alkylated such as ethylated, butylated, modified with butyl cellosolve, or the like; those having a maleic acid side chain of which the carboxyl group is amidated, aminoethylated, trishydroxyaminoethylated, hydroxyaminomethanized, mercaptoethylaminated, polyethylene-glycolated (PEG), or amino acidified (e.g., lysine, cysteine, other amino acid conjugates, and the like); and those having a maleic acid side chain modified with hydrazine.

Examples of the SMA in which the carboxyl group in the side chain is butylated or butylcellsolvated include SMAR Resins (Sartomer, Kawahara Petrochemical Co., LTD.) and the like.

The SMA derivative in the present invention also includes SMA derivatives disclosed in WO2015/076312, for example, the following SMA derivatives:

(1) A SMA derivative comprising a side chain containing a functional group selected from —NH₂, —SH, —OH, —COOH, —NH—(C═NH) —NH₂ and —C(CH₂—OH)₃, which is introduced into the half of dicarboxyl group in the maleic acid residue in SMA via an amide bond, an ester bond, or a hydrazone bond;
(2) The SMA derivative according to the above [1], wherein the side chain (b) is represented by the following formula [A]:

—C(═O)—NH—R¹—R² [A]

wherein R¹ is a group selected from a single bond, an alkylene group, —NH—, —CO—, —(C═NH)—, —N═C(CH₃)—, —(C═NH)—NH—, and a combination thereof, wherein the alkylene group is optionally substituted by a group selected from a hydroxyl group and a carboxyl group;
R² is a group selected from a hydrogen atom, —NH₂, —SH, —OH, —COOH, —NH—(C═NH)—NH₂ and —C(CH₂—OH)₃, provided that when R² is a hydrogen atom, R¹ is a single bond;
wherein when the SMA derivative includes a plurality of groups represented by the formula [A], each R¹ and R² may be identical or different.
(3) The SMA derivative according to the above (2), wherein R¹ in the formula [A] is selected from a single bond, —CH₂—, —(CH₂)₂—, —(CH₂)₃—, —CH(COOH)—CH₂—, —CH₂—CH (COOH)—, —CH₂—CH(OH)—CH₂—, —(CH₂)₄—, —CH(COOH)—(CH₂)₃—, —(CH₂)₃—CH(COOH)—, —(CH₂)₃—CO—CH(COOH)—, —CH₂—CO—(CH₂)₂—, —N═C(CH₃)—(CH₂)₂—, —(CH₂)₅—, —CH(COOH)—(CH₂)₄—, —(CH₂)₄—CH(COOH)—, —(CH₂)₄—NH—(C═NH)−, —(C═NH)—NH—(CH₂)₄—, —CH(COOH)—(CH₂)₃—NH—(C═NH)—, —(C═NH)—NH—(CH₂)₃—CH(COOH)— and —(CH₂)₆—, and these groups having a ketone group on the α, β, γ, or δ carbon atom of carboxyl group.
(4) The SMA derivative according to the above (2) or (3) wherein —R¹-R² in the formula [A] is selected from the following groups:

(1) a hydrogen atom,

(2) —NH₂,

(3) —(CH₂)₂—SH,

(4) —CH(COOH)—CH₂—SH,

(5) —(CH₂)₁₋₆—NH₂,

(6) —CH₂—CH(OH)—CH₂—NH₂,

(7) —CH(COOH)—(CH₂)₄—NH₂,

(8) —(CH₂)₁₋₄—CH(COOH)—NH₂,

(9) —(CH₂)₁₋₄—NH—(C═NH)—NH₂,

(10) —(C═NH)—NH—(CH₂)₁₋₄—NH₂,

(11) —CH(COOH)—(CH₂)₃—NH—(C═NH)—NH₂,

(12) —(C═NH)—NH—(CH₂)₃—CH(COOH)—NH₂,

(13) —C(CH₂—OH)₃,

(14) —(CH₂)₁₋₄—NH—CO—NH—NH₂,

(15) —(CH₂)₁₋₄—CO—CH₂—NH₂,

(16) —CH₂—CO—(CH₂)₄—NH₂,

(17) —CH₂—CO—(CH₂)—OH,

(18) —(CH₂)₁₋₄—CO—CHOH—COOH,

(19) —CH₂—CO—(CH₂)₂—COOH,

(20) —N═C(CH₃)—(CH₂)₂—COOH,

(21) —(CH₂)₃—NH₂, and

(22) —(CH₂)₃—OH.

In the present invention, the SMA (including derivatives thereof hereinafter unless specifically referred to as “derivative”.) can be used alone or as a mixture of two or more alone.

The SMA used for the macromolecular complex of the present invention may have various molecular weights depending on the polymerization degree. For example, the SMA may have a polymerization degree (n) of about 3-500, an apparent weight average molecular weight (Mw) in an aqueous solution of from about 500 to 100,000 Daltons (Da), preferably from about 1,000 to 5,000 Da.

Here, the apparent weight average molecular weight (Mw) of SMA can be measured by a static light scattering method (SLS) using a multi-angle light scattering detector as described later.

The “boric acid compound” is not particularly limited as long as it contains a boric acid structure. Examples of the boric acid compound include boric acid, disodium tetraborate (Borax, Naₑ tetraborate decahydrate, Na₂BO₄.H₂O), etc.

In the present invention, the boric acid compound can be used alone or as a mixture of two or more alone.

The boron atom constituting the boric acid compound is preferably one enriched in the isotope of ¹⁰B atom, which is more effective for BNCT, namely, [¹⁰B]>[¹¹B].

In the complex of the present invention, the SMA and the boric acid compound are bound directly or via a linker. Hereinafter, a complex wherein the SMA and the boric acid compound are directly bonded is referred to as “SMA-B”, and a complex wherein the SMA and the boric acid compound are bound via a linker is referred to as “SMA-L-B”.

Examples of the complex (SMA-B) wherein the SMA and the boric acid compound are directly bound include those having the following structure (2):

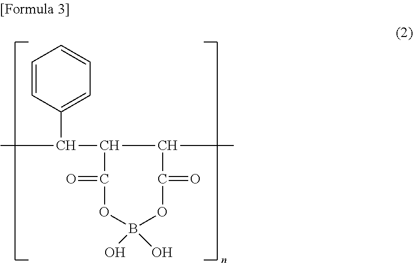

In the formula (2), n represents an integer of 2 or more, for example, 3 to 500.

The above SMA-B can be prepared by slowly adding a boric acid compound (for example, boric acid, borax, or the like) to an aqueous solution of SMA (pH 8 to 9) under stirring for 10 to 40 hours to bind the boric acid compound to the SMA.

The reaction temperature in this reaction is, for example, about 20 to 60° C., preferably about room temperature (20 to 30° C.), and the reaction time is, for example, about 10 to 40 hours, preferably about 24 hours. Further, this reaction is preferably carried out in a 3 to 20% aqueous solution of SMA.

The amount of the boric acid compound used in this reaction is not particularly limited, but is preferably an excess amount relative to SMA, for example, 1 to 100 molar equivalents, preferably from 1 to 5 molar equivalents, relative to the maleic anhydride residues of SMA.

The linker in a complex where the SMA and the boric acid compound are bound via a linker (SMA-LB) includes those having a functional group (a) for binding to the SMA and a functional group (b) for binding to the boric acid compound.

The functional group (a) is not particularly limited as long as it can covalently bind to the SMA, but includes preferably a functional group capable of forming a covalent bond with a carboxyl group in the maleic acid residues of SMA. Specific examples of such functional group (a) include an amino group (—NH₂), a hydroxyl group (—OH), a thiol group (—SH), a hydrazine group (—NH—NH₂), etc., and preferably an amino group.

The functional group (b) is not particularly limited as long as it can covalently bind to the boric acid compound, but preferably includes a hydroxyl group and the like, and more preferably two adjacent (cis) hydroxyl groups (for example, —(CH)₂₋₃—(OH)₂₋₃, e.g., cis-diol group) and the like.

Examples of the linker include sugars, amino sugars, sugar alcohols, and the like, specifically glucosamine, glucose, chitin, chitosan and the like, particularly those having cis-diol group (cis-diol compounds) such as α-D-glucopyranose, α-D-ribofuranose, α-D-erythrose, glyceraldehyde, and the like, preferably glucosamine and the like.

The linker in the present invention, per se, preferably has anticancer effects. Such linker includes, for example, glucosamine, 5-fluorouracil, analogs of nucleic acid, and the like.

In addition, it has been already reported that glucosamine exhibits an anticancer activity (for example, Cancer Cell International, 14: 45 (2014), Mol. Med. Rep. 16: 3395-3400 (2017), PLOS ONE 13 (7): e0200757 (2018)).

Further, in the present invention, the above linker may be a functional group capable of bonding with a boric acid compound, which constitutes a SMA derivative (introduced into SMA).

In the SMA-L-B, the linker is preferably bound to SMA via an amide bond, an ester bond, a thioester bond, or a hydrazone bond. These bonds are formed by a reaction of a carboxyl group in the maleic acid residues of SMA with an individual functional group (a) of the linker, i.e., an amino group (—NH₂), a hydroxyl group (—OH), an thiol group (—SH), or a hydrazine group (—NH—NH₂), respectively.

The SMA-L-B can be produced, for example, by a method including the following steps:

(a) binding the linker to the SMA,
(b) binding a linker residue in the product obtained in the step (a) to the boric acid compound.

Hereinafter, a method for producing the complex of the present invention will be explained in more detail with reference to an example wherein the SMA is SMA (styrene-maleic anhydride copolymer), the boric acid compound is boric acid, and the linker is a linker having an amino group as the functional group (a) and two adjacent hydroxyl groups as the functional group (b).

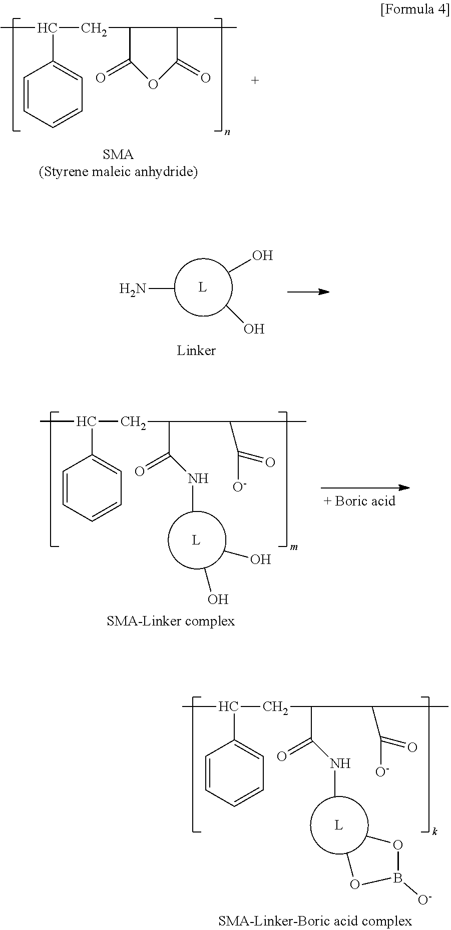

wherein, n, m and k are each independently an integer of 2 or more, for example, 3 to 500, and satisfy n≥m≥k, and the repeating unit represented by [ ] may not be continuous. Further, L is a linker portion other than the functional groups (a) and (b).

Firstly, the amino group (—NH₂) of the linker (e.g., glucosamine) is reacted with the maleic anhydride residue of SMA to obtain a SMA-Linker conjugate wherein the linker residue is hanging from SMA chain like a pendant.

The reaction temperature in this reaction is, for example, about 10 to 70° C., preferably about 50 to 55° C., and the reaction time is, for example, about 5 to 50 hours, preferably about 24 hours. The present reaction is preferably carried out in an aqueous solution at pH 8 to 9.

The amount of the linker used in this reaction is not particularly limited, but is, for example, 1 to 50 molar equivalents, preferably 2 to 10 molar equivalents, relative to the maleic anhydride residue.

The SMA-linker conjugate obtained in the above step can be purified as needed. The purification method is not particularly limited, and may be carried out by known methods. For example, the complex can be purified by solubilizing the complex (precipitate) with an alkaline water having pH 7 to 8, concentrating the solution with dialysis or ultrafiltration against distilled water, and repeating these steps. Also, the complex can be lyophilized after purification.

Then, to an aqueous solution of the SMA-linker conjugate is slowly added boric acid under stirring for 10 to 40 hours to bind the boric acid to the SMA-linker conjugate to obtain a SMA-linker-boric acid complex.

The reaction temperature in this reaction is, for example, about 20 to 60° C., preferably about room temperature (20 to 30° C.), and the reaction time is, for example, about 10 to 40 hours, preferably about 24 hours. Further, this reaction (incorporation of boric acid) is preferably performed in a 3 to 20% aqueous solution of the SMA-linker conjugate.

The amount of boric acid used in this reaction is not particularly limited, but is preferably an excess amount relative to the linker residue, for example, 1 to 100 molar equivalents, preferably 1 to 5 molar excess equivalents, relative to the linker residue.

In the above process, disodium tetraborate can be similarly used instead of boric acid.

The SMA-linker-boric acid complex (SMA-L-B) obtained in the above steps can be purified as needed. The purification method is not particularly limited, and may be carried out by known methods. For example, the complex can be purified by solubilizing the complex (precipitate) with an alkaline water of pH 7 to 8, concentrating the solution with dialysis or ultrafiltration against distilled water, and repeating these steps. Also, the complex can be lyophilized after purification.

The apparent molecular weight of SMA-boric acid complex (SMA-B) and SMA-linker-boric acid complex (SMA-L-B) of the present invention is not particularly limited, but the apparent weight average molecular weight (Mw) in an aqueous solution is, for example, 5 kDa to 200 kDa, preferably 5 kDa to 100 kDa, particularly 10 kDa to 100 kDa.

Here, the apparent weight average molecular weight (Mw) of the complex of the present invention can be measured by a static light scattering method (SLS) using a multi-angle light scattering detector as described later.

The average particle size of the complex of the present invention is not particularly limited, but is, for example, 3 to 200 nm in diameter, preferably 5 to 100 nm in diameter.

Here, the average particle size of the macromolecular complex of the present invention can be measured by a dynamic and static light scattering method in 0.1 M tris-HCl buffer (pH 8.2) as described below.

The amount of boric acid compound (e.g., boric acid) bound to the macromolecular complex of the present invention is not particularly limited, but is, for example, 3 to 30% (w/w), preferably 5 to 15% (w/w).

Here, the boric acid binding amount can be measured by the method described in the document: J. T. Hatcher and L. V. Wilcox. Colorimetric determination of boron using carmine. Anal. Chem. 22 (4), 567-569, 1950, as described later.

BNCT is a method for the treatment of cancer, which comprises administering a boron (¹⁰B)-containing formulation to a patient, and then irradiating the tumor sites with neutrons (thermal neutrons) which were generated by accelerators or nuclear reactors to generate a ray that is the main cell killing factor. The complex of the present invention is a macromolecule containing boron that can be used for BNCT, which is obtained by using a boric acid compound and a polymer. The complex of the present invention exhibits the EPR effect in vivo, and thus preferentially accumulates in tumor sites. For example, after 24 hours of intravenous injection, the accumulation of the complex in tumors is 20 times or more superior to that of normal tissues. According to the complex of the present invention, it is thus possible to allow a larger amount of boron to be accumulated at tumor sites than other sites, and thus the therapeutic effect (anticancer effect) of BNCT can be significantly improved while the side effects can be reduced in other sites than tumor sites. Therefore, the complex of the present invention is much more beneficial as an anticancer agent, particularly for BNCT, as compared to conventional small molecular anticancer agents.

Conditions for BNCT using the complex of the present invention are not particularly limited, and known conditions can be used.

Also, the complex of the present invention is capable of releasing the free boric acid compound in an acidic pH. This is proved in the examples described later. Here, the majority of solid tumors depend on anaerobic fermentation, i.e., glycolytic system of glucose, for the energy (ATP) necessary for surviving. A free boric acid compound can inhibit the first step of phosphorylation of glucose in this glycolytic system (Warburg effect) and thus can suppress the energy production (ATP) and the growth of cancer cells. Since tumor sites exhibit an acidic pH, the complex of the present invention can release a free boric acid compound at the tumor sites to suppress the growth of cancer cells. That is', the complex of the present invention may be useful as an anticancer agent in addition to the usability for BNCT.

Therefore, the complex of the present invention may exert anticancer effects in two mechanisms of inhibition of glycolytic system as well as therapeutic effect by BNCT.

The complex of the present invention can also inhibit glucose uptake into cells. Therefore, the complex of the invention can be used as an inhibitor of glucose uptake into cells, and can be used for diseases whose symptoms can be improved by it. Examples of the diseases include colon cancer, pancreatic cancer, breast cancer, brain tumor, gall bladder cancer, deep infection, pneumonia, and conjunctivitis.

Further, the above SMA-linker conjugate itself can be used as an anticancer agent, if the linker itself has an anticancer effect. Examples of such SMA-linker conjugates include conjugates of SMA and glucosamine (SMA-glucosamine conjugate, SG), conjugates of SMA and 5-fluorouracil, conjugates of SMA and an analog of nucleic acid, and the like.

The conjugate SG is a macromolecule containing glucosamine and thus accumulates more at the tumor sites than at other sites by the EPR effect. Then, the conjugate is slowly cleaved by hydrolases/proteases/amidases in tumor cells to release glucosamine, which exerts anticancer activity. Thus, the conjugate itself is useful as an anticancer agent.

Therefore, the complex of the present invention comprising a linker having an anticancer effect (e.g., glucosamine) can exhibit a better anticancer effect, since the complex is dissociated in vivo to release not only a boric acid compound but also the linker.

The complex and the SMA-linker conjugate of the present invention can bind to albumin, because it contains SMA. The binding of SMA to albumin has been reported in, for example, Tsukigawa et al, Cancer Science, 106, 270-278 (2016), and is also proved in the examples described later.

Therefore, the complex and the conjugate of the present invention exist in vivo (i.e., in blood) in a size to which the molecular size (about 70 kDa) of albumin is added, which is advantageous for exerting the EPR effect. Therefore, the complex of the present invention is very useful as an anticancer agent, particularly as an anticancer agent for BNCT, since it can accumulate more selectively in the tumor sites. Similarly, the conjugate of the present invention is useful as an anticancer agent.

In addition, the complex of the present invention exhibits excellent antibacterial activity against both Gram-positive and Gram-negative bacteria, and thus the complex can be advantageously used as antibacterial agents and can be used as agents for the treatment or prevention of infections caused by these bacteria. The complex of the present invention can be used in various formulations as an antibacterial agent for the treatment or prevention of infections caused by, for example, beta-lactam-resistant bacteria and MRSA.

The complex or the conjugate of the present invention can be safely administered to a mammal (e.g., mouse, rat, hamster, rabbit, cat, dog, cow, sheep, monkey, and human) and can be used for the prevention or treatment of various diseases (especially cancer) in patients (mammals). Accordingly, the present invention provides a method for treating or preventing various diseases (particularly cancer), which comprises administering the complex or the conjugate of the present invention to a patient.

Furthermore, the complex of the present invention can be used for boron neutron capture therapy (BNCT), and thus the present invention also provides a method for treating cancer, which comprises administering the complex of the present invention to a patient (mammal) suffering from cancer, and then irradiating the tumor sites with neutrons (thermal neutrons) generated by accelerators or nuclear reactors.

In addition, the conjugate of the present invention can be used as an anticancer agent, and thus the present invention also provides a method for treating cancer, which comprises administering the conjugate of the present invention to a patient (mammal) suffering from cancer.

The present invention also provides a pharmaceutical composition comprising the complex or conjugate of the present invention and a pharmaceutically acceptable carrier. The pharmaceutically acceptable carrier may be any conventionally used in the field of drug formulation, and is not particularly limited.

In addition, the present invention provides the complex or conjugate of the present invention for use in the treatment or prevention of various diseases (e.g., cancer).

The present invention also provides the use of the complex or conjugate of the present invention for the manufacture of a medicament for the treatment or prevention of various diseases (e.g., cancer).

When the complex or conjugate of the invention is used as a medicament, the usage, dosage, amount, formulation, etc.

are not particularly limited and can be determined according to the target disease, patient, dosage form, etc.

### EXAMPLES

### Example 1: Synthesis of SMA-Glucosamine-Boric Acid Complex

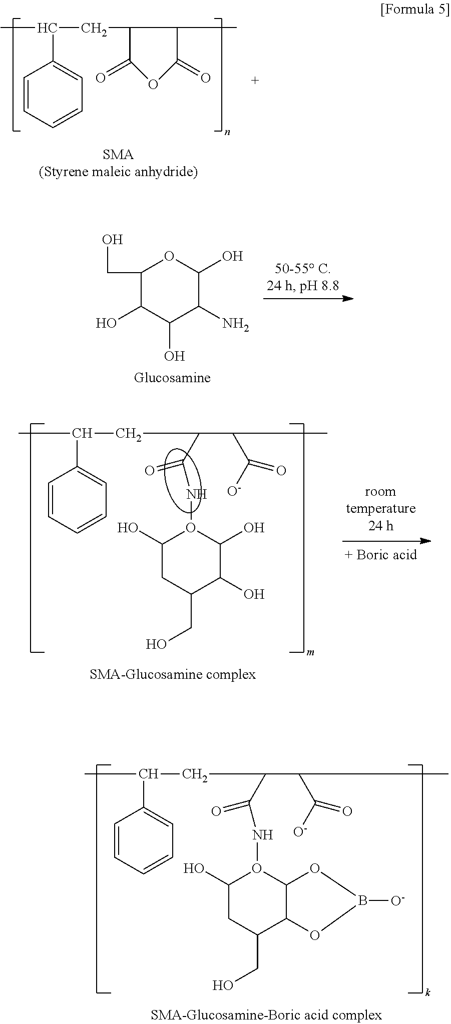

According to the above reaction scheme, a SMA-glucosamine-boric acid complex was synthesized. Specifically, the following steps were performed.

### (1) Synthesis of SMA-Glucosamine Conjugate

To a SMA (styrene-maleic anhydride copolymer, the apparent weight average molecular weight thereof in aqueous solution is 7,500 Da, Sortmer®, Kawahara Petrochemical Co., LTD.) in 0.2 M sodium bicarbonate solution (pH 8.8) was added glucosamine (Wako Pure Chemical Industries, Ltd.) at a 50-fold molar excess of glucosamine relative to the maleic anhydride under stirring, and a conjugation reaction of amino group with a maleic anhydride was allowed to proceed at 50 to 55° C. When the pH became 7.5 or less, sodium carbonate was added to the solution to adjust the pH to 8.5 or above, and then the reaction was continued. The reaction solution became clear after 24 hours, and then it was dialyzed against pure water to remove unreacted glucosamine. The external dialysis water was replaced with pure water five times every six hours. The dialyzed solution was lyophilized to obtain a powdered SMA-glucosamine conjugate (SG). The yield was about 80% (w/w) relative to the SMA.

This lyophilized SG was attempted to be dissolved in distilled water to obtain a 10 mg/ml solution, but it was poorly soluble. When the SG was dissolved in 0.2 M sodium bicarbonate solution, the pH was 8.5.

In addition, when the SG was dialyzed against distilled water, lyophilized, and dissolved in distilled water at a concentration of 10 mg/ml, the pH was 6 to 8.

### (2) Synthesis of SMA-Glucosamine-Boric Acid Complex

The SMA-glucosamine conjugate (SG, 100 mg) obtained in the above step (1) was dissolved in 0.2 M sodium bicarbonate solution (pH 8.8). To the solution was added a 50 molar excess of boric acid and stirred with a magnetic stirrer to dissolve it. After the solution was left standing at room temperature for 24 hours, it was dialyzed against pure water for 24 hours to remove a low molecular boric acid in the same manner as in the above step (1). The external dialysis water was replaced with pure water five times. The dialyzed solution was lyophilized to obtain a SMA-glucosamine-boric acid complex (SGB). The yield was about 90% (w/w) relative to the SG. When the lyophilized SGB was dissolved in distilled water at a concentration of 10 mg/ml, the pH was about 7.5.

The boric acid content contained in the obtained SGB was quantified according to the method described in the literature: J. T. Hatcher and L. V. Wilcox. Colorimetric determination of boron using carmine. Anal. Chem. 22 (4), 567-569, 1950.

That is, firstly, standard solutions of boric acid (manufactured by Wako Pure Chemical Industries, Ltd.) at a concentration of 0.1 to 1.0 mg/ml were prepared, and 1 ml of each solution was used as standard solution. To each test tube containing each standard amount of boric acid were added 2 to 3 drops of concentrated hydrochloric acid and 0.5 ml of concentrated sulfuric acid, and then mixed well and cooled. Then, to each test tube was added 0.5 ml of a 0.05% solution of carmine in concentrated sulfuric acid, stirred well, and left standing for 45 minutes or more. A calibration curve was generated based on the appearing red color (absorption at 545 nm). The amount of boric acid in the SMA-glucosamine-boric acid complex was quantified based on a calibration curve of standard boric acid solution. As a result, this complex contained about 7.3% (w/w) of boric acid.

### Analysis of IR Absorption Spectrum

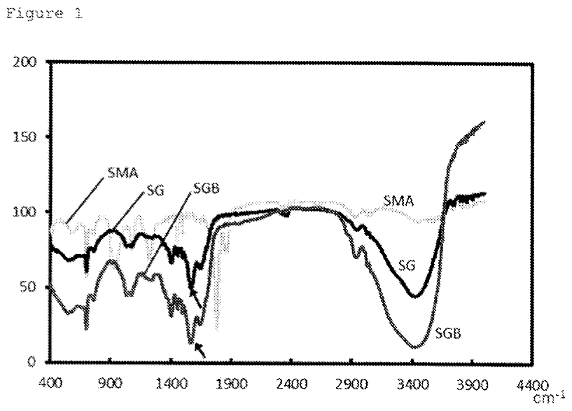

About 1 mg of a powder of each of the SMA, the SMA-glucosamine conjugate (SG) and the SMA-glucosamine-boric acid complex (SGB) was taken and mixed well with about 200 mg of a powder of KBr. The mixture was thoroughly dried in a vacuum in the presence of P₂O₅. After that, it was prepared into a pellet under pressure by a conventional method and determined the Fourier infrared absorption spectrum thereof. As a result, an intrinsic peak of amide bond (—CO—NH—) that was formed by condensation reaction of a carboxyl group (—COOH) of SMA with an amino group (—NH₂) of glucosamine was detected for the complexes (SG and SGB) (FIG. 1).

### Measurement of UV Absorption Spectrum

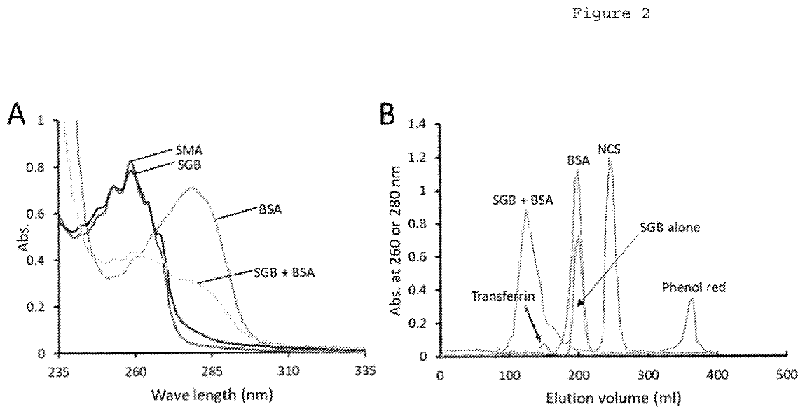

The ultraviolet absorption spectra (wavelengths 235 to 310 nm) of SMA and SGB were measured. For comparison, spectra of human transferrin, bovine serum albumin (BSA), neocarzinostatin (NCS), and a mixture of SGB and BSA (SGB+BSA) were measured. The results are shown in FIG. 2A.

### Analysis by Gel Permeation Chromatography (GPC)

SGB was dissolved in 0.1 M tris-HCl buffer (pH 8.2) at a concentration of 10 mg/ml. To 1 ml of the solution was added BSA to obtain a 3% solution, or an additive-free solution was prepared, and then each solution was left standing at room temperature for about 5 hours. After that, each solution was subjected to a chromatography on Sephacryl S-300 column (2 cm×60 cm, GE Healthcare) and eluted at a rate of 0.4 ml/min, and 4.0 ml of each fraction was collected and monitored the elution of the active ingredient by measuring the absorption at 260 nm and 280 nm, where for elution buffer, 0.1 M tris-HCl buffer (pH 8.2) was used. Human transferrin (90 kDa), BSA (67 kDa) and NCS (12 kDa) were used as the molecular weight standards. The results are shown in FIG. 2B.

The apparent molecular weight of the active ingredient (SGB+BSA) in a mixed solution of SGB and BSA was about 150 kDa. Since the molecular weight of the original SGB was about 65 kDa and that of BSA was about 67 kDa, it thus shows complex formulation of SGB and BSA.

### Measurement of Molecular Weight by Static Light Scattering Method

The apparent weight average molecular weight of SMA, SG and SGB before the reaction was measured by static light scattering (SLS) method with a multiangle light scattering detector (DAWN HELEOS II using) manufactured by Wyatt Technology Corp. Santa Barbara, Calif., USA. The results are shown in Table 1.

### Measurement of Particle Size by Dynamic Light Scattering Method

Each of SMA, SG and SGB before the reaction was dissolved in 0.1 M tris-HCl buffer (pH 8.2) at a concentration of 15 mg/ml and filtrated through a 0.2 pm filter (which was connected to a syringe : Millipore Company, Ltd.). Then, each solution was measured at 25° C. by a light scattering apparatus, Model ELSZ⋅2000ZS manufactured by Otsuka Electronics (Photal Inc., Osaka). The apparatus uses a He/Ne laser as a light source and displays data as a histogram. The results are shown in Table 1.

### Measurement of Particle Size of the Above Boric Acid Complex by Transmission Electron Microscope (TEM)

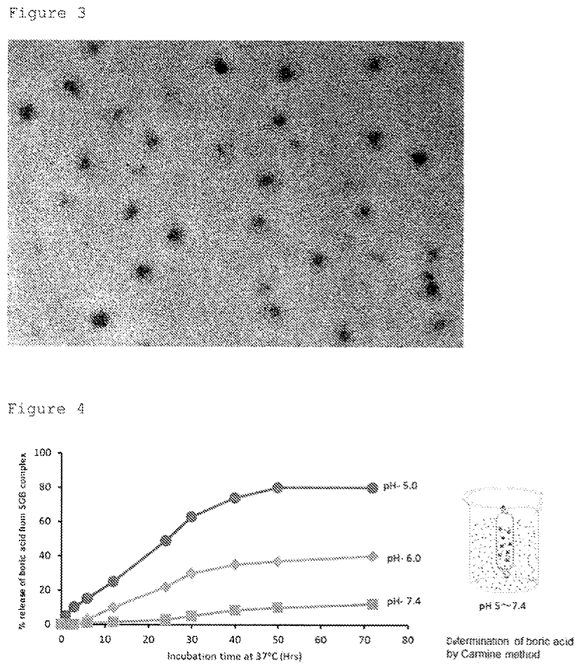

The above complex was dissolved in distilled water at a concentration of 10 mg/ml, and 50 μl of the solution was taken into a micro test tube. To the micro test tube was added 50 μl of 0.1% phosphotungstic acid and the mixture was stirred. About 10 μl of the solution was attached to the metal grid of a transmission electron microscopy (TEM) (ELS-C10, Okenshoji Co., Ltd), and then the grid was placed in a desiccator and dried under vacuum for more than overnight. The grid was loaded into a TEM (JEOL, JEM-1400 Plus, Tokyo, Japan) by a conventional method, and the particles were observed by TEM. The results are shown in Table 1 and FIG. 3. The average size of these particles was 85±5.5 nm (average of 30 particles).

### Measurement of Surface Charge (Zeta Potential)

Each product of SMA (before each production step), SG and SGB was dissolved in deionized water at a concentration of 20 mg/ml and then the surface charge (Zeta potential) was measured. The addition of glucosamine decreased the negative surface charge of SMA from −47.5 mV to −27 mV due to incorporation of amino group. By the addition of boric acid to this SG, the negative charge of boric acid added to SG increased to −37 mV. The results are shown in Table 1.

TABLE 1
Characteristics of SMA, SMA-glucosamine conjugate
(SG) and SMA-glucosamine-boric acid complex (SGB)

| | | | | | SMA- | |
|---|---|---|---|---|---|---|
| | | | SMA- | | glucosamine- | |
| | | | glucosamine | | boric acid | |
| | SMA | | conjugate(SG) | | complex(SGB) | |
| Color | White | | Light yellow | | Off-white | |
| Apparent weight | 7,500 | | 8,000 | | 10,000 | |
| average molecular | | | | | | |
| weight (Wyatt | | | | | | |
| Technology Corp., | | | | | | |
| static light | | | | | | |
| scattering (SLS)) | | | | | | |
| Intrinsic pH in | 4.5 | | 8.5 | | 7.2 | |
| solution of | | | | | | |
| lyophilized product | | | | | | |
| in distilled water | | | | | | |
| Average particle | 60 ± 5 | nm | 90 ± 3 | nm | 95 ± 5 | nm |
| size (dynamic light | | | | | | |
| scattering method) | | | | | | |
| in diameter | | | | | | |
| Average particle | — | | — | | About 85 nm | |
| size by | | | | | | |
| transmission | | | | | | |
| electron microscope | | | | | | |
| (dried state) | | | | | | |
| Surface charge | −47.5 | mV | −27 | mV | −37 | mV |
| (Zeta potential) | | | | | | |

### Test Example 1: Release of Boric Acid From SMA-Glucosamine-Boric Acid Complex

The pH of tissues of solid tumors is known to be weakly acidic (pH 5 to 6.5 vs normal tissue pH 7.4). On the other hand, energy (ATP) generation in advanced solid tumors mainly relies on glycolytic metabolic system utilizing glucose. Free boric acid competitively inhibits the glycolytic system to free glucose (Warburg effect), thereby resulting in the suppression of tumor growth.

This test is to confirm that free boric acid (BO₃⁻³) is generated from a SMA-glucosamine-boric acid complex at a weakly acidic pH of solid tumors as represented by the following formula.

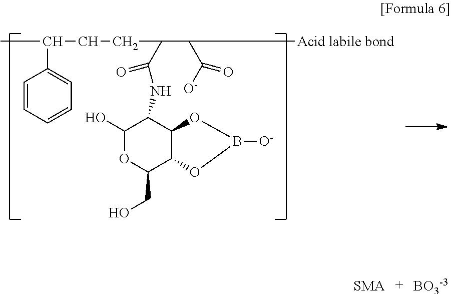

Firstly, 100 mg of the SMA-glucosamine-boric acid complex (SGB) obtained in Example 1 was dissolved in 1 ml of each buffer at pH 5.0, 6.0, and 7.4. Each solution was added to a dialysis tube (Visking, p10 mm) with a cut-off value of about 6 KDa and dialyzed against the same buffer in a large test tube of 20 ml at 37° C. under shaking, and the release of boric acid was monitored over time. Each external dialysis solution was 20 ml of the same buffer. A sample of 0.5 ml of the external solution was taken over time and the amount of free boric acid was determined by Carmine method. The results are shown in FIG. 4. From the results, it was found that boric acid was the most quickly released at acidic pH 5.0.

### Test Example 2: Bioactivity of SMA-Glucosamine-Boric Acid Complex In Vitro

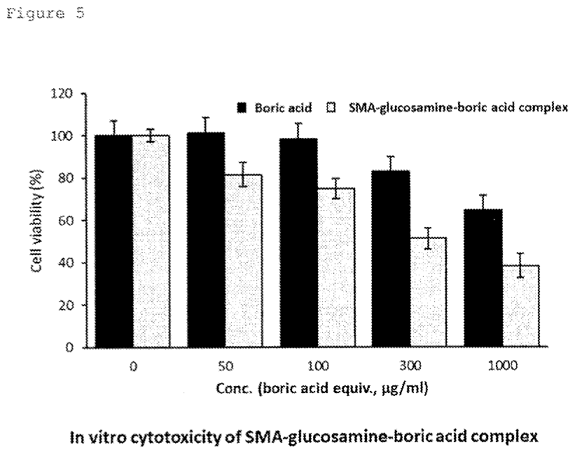

The cytostatic effect of the above complex (SGB) was examined for cell viability by MTT assay (tetrazolium salt, manufactured by Dojindo Laboratories, Kumamoto) using HeLa cells (1×10⁴/well) in Falcon 96-well plastic plate (BD Labware, Franklin Lake, NJ, USA). The culture of cells was performed in an Eagle MEM medium supplied with 10% fetal calf serum at 37° C. in 95% air/5% CO₂. To this system was added free boric acid or the above SMA-glucosamine-boric acid complex, and then the culture was continued at 37° C. for 24 hours. After that, the medium was replaced with a fresh medium and the culture was performed for further 24 hours. Then, viable cells was measured by the MTT method. The results are shown in FIG. 5. Although the glycolytic system of solid tumors in vivo are mostly increased under anaerobic conditions of advanced tumor tissues, this culture was conducted under aerobic conditions. Thus, the obtained data showed relatively lower values, but it was found that the growth of cancer cells was significantly suppressed.

### Test Example 3: In Vitro Cytotoxicity of Glucosamine, SMA-Glucosamine Conjugate and SMA-Glucosamine-Boric Acid Complex on Mouse Colon Cancer Cells (C26)

These C26 cells subcultured intraperitoneally in mice were washed with a MEM medium (centrifugation, 1,500 rpm) and then adjusted the number of cells to 10⁵/ml. The cells in 0.1 ml (MEM medium) was cultured according to Test Example 2 (the number of cells: 1×10⁴ cells/well), except for using a medium containing 0.1% (normal concentration) glucose, or a medium containing about 0.01% glucose, which was obtained by adding 10% FCS (fetal bovine serum, Gibco) to a glucose-free medium, under a low oxygen partial pressure of 6 to 9% (vs normal pressure of 21%) that simulates the microenvironment of advanced human solid cancer tissues.

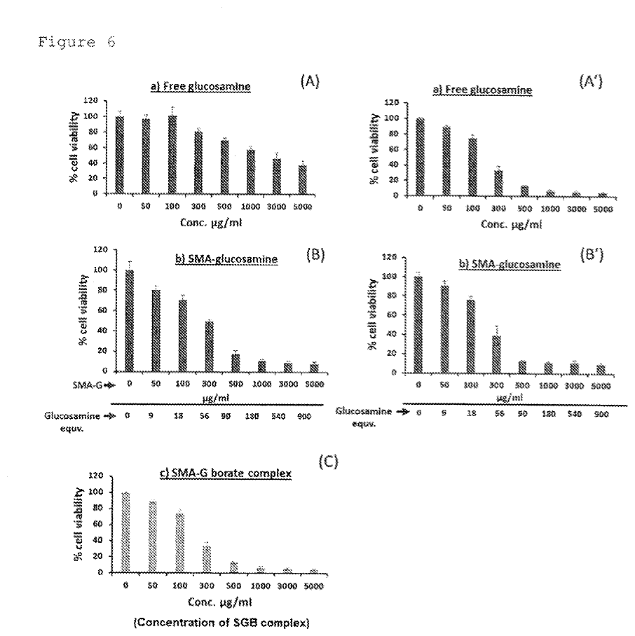

Firstly, the above cells were cultured at a normal oxygen partial pressure for 24 to 30 hours, and then the culture medium [Petri dish, 96 wells] was transferred to a microaerobic conditions, which was made by adding a commercially available oxygen absorber to an anaerobic culture chamber, i.e., a hypoxic agent (oxygen absorber), AnaeroPack—Micro aero generator manufactured by Mitsubishi Gas Chemical Company, Inc., Tokyo, was added into an anaerobic culture chamber to be a microaerobic (hypoxic) oxygen partial pressure (6 to 9%). The sealed chamber used in this test was a medium square jar (3.5 L) manufactured by Sugiyama-Gen Co., Ltd., Tokyo. In this state, the reagents to be examined were added to each medium at a predetermined concentration, and further cultured for 36 hours. After that, the number of living cells was measured by the above MTT method. The results are shown in FIG. 6.

FIGS. 6(A), (B) and (C) show the results where C26 cells were cultured in an ordinary medium containing 0.1% glucose and FIGS. 6(A′) and (B′) show the results where the cells were cultured in a medium containing almost no glucose (0.01%) at a low pH and a low oxygen partial pressure, which conditions correspond to tumor sites.

Free glucosamine was used as a drug in FIGS. 6(A) and (A′), the SMA-glucosamine conjugate (SG) was used as a drug in the (B) and (B′), and the SG-boric acid (SGB) complex was used as a drug in the (C).

As shown in FIG. 6, the cytostatic action was strengthened in the order of (A) free glucosamine, (B) SG and (C) SGB. When mouse colon cancer cells C26 was cultured in a medium containing no glucose ((A′) and (B′)), which is similar to no (low) glucose-condition in clinical solid cancers, the cytotoxic effect of glucosamine was 2 to 5 times stronger than those obtained in a glucose-supplemented medium ((A), (B) and (C)).

### Test Example 4: Comparison of the In Vitro Cytotoxicity of Each Drug in C26 and HeLa Cells Under Normoxic and Hypoxic Partial Pressures for 48 Hours

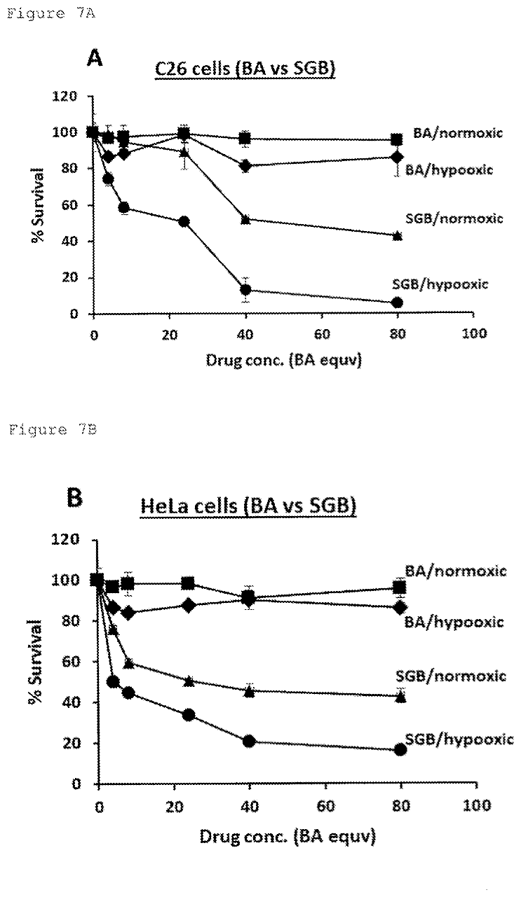

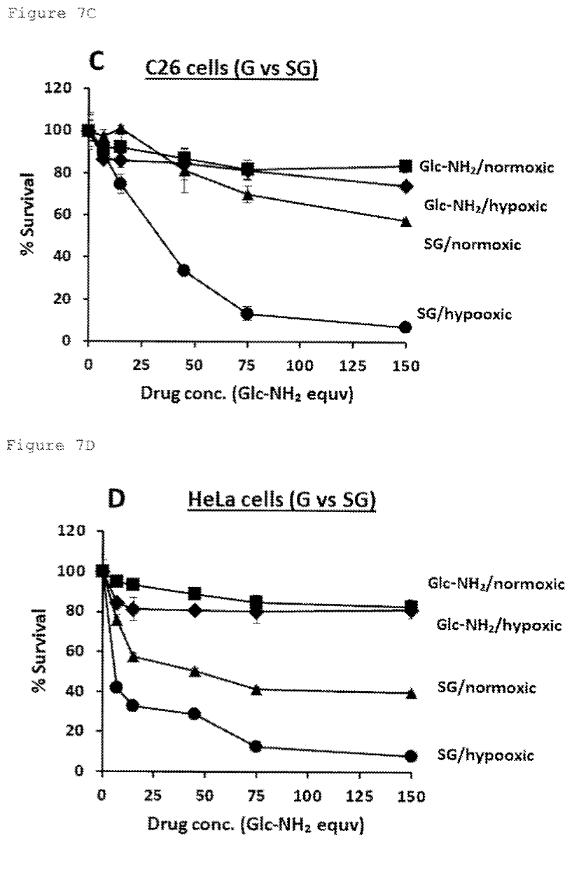

Colon cancer C26 and HeLa cells (1×10⁴ cells/well) were seeded in Falcon 96-well culture plates and cultured overnight at 37° C. in Eagle MEM containing 10% FBS under normoxic partial pressure (5% CO₂, 95% air) and hypoxic partial pressure (using a hypoxic chamber, pO₂ 6-8%). Both C26 and HeLa cells were treated in the presence of boric acid (BA) or SGB and cultured for 48 hours under normoxic or hypoxic partial pressures. Finally, cell viability was analyzed by MTT assay. The results are shown in FIGS. 7A and 7B.

C26 and HeLa cells were also cultured as described above (FIGS. 7A and 7B) and treated with glucosamine (G) and SMA-glucosamine (SG). Finally, cell viability was measured by MTT assay.

From these figures, it is clear that SG and SGB exhibit very potent cytotoxicity, especially under low oxygen partial pressures similar to the environment of solid cancer tissues.

### Test Example 5: In Vivo Toxicity Assessment of SMA-Glucosamine-Boric Acid Complex

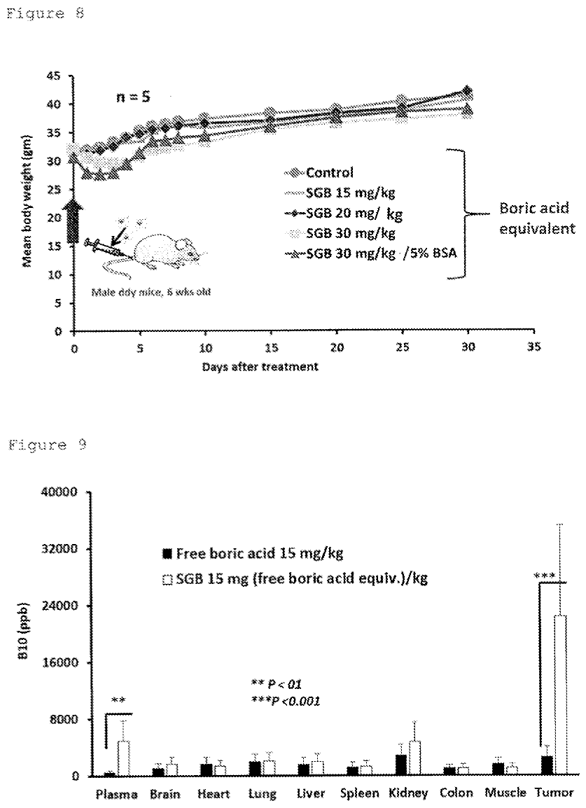

The in vivo activity assay (toxicity assessment) was performed by using 6-week-old male ddY mice. Firstly, the SMA-glucosamine-boric acid complex (SGB) was dissolved in saline to obtain 15, 20, and 30 mg/kg (boric acid equivalent) solutions. Then, 0.1 ml of each solution was intravenously administered to the mice. The content of boric acid in SGB used was 7 to 8% (w/w). The body weight and other indicators were monitored for 30 days after the administration. The results are shown in FIG. 8.

In the group of 30 mg/kg (boric acid equivalent) administration, there was a little weight loss on 2 to 4 days, but it was recovered on 5 to 6 days. No serious toxicity was shown in this group. The total dose of SGB was 375 mg/kg based on the polymer conjugate (wt)/body (wt), which corresponds to 13.2 mg/mouse.

### Test Example 6: Comparison in Distribution After Intravenous Injection Between Free Boric Acid and SMA-Glucosamine-Boric Acid Complex

Mouse S180 tumor (solid type, sarcoma) cells were implanted (10⁶) subcutaneously on the back of mice. When the diameter of the tumor became about 10 to 12 mm, the SMA-glucosamine-boric acid complex (SGB) containing boron was administered by intravenous injection. In this test, the amounts of the original boric acid and the above SGB were expressed as a boric acid equivalent. They were dissolved in distilled water to obtain a 15 mg/kg solution, respectively, and about 0.1 ml of each solution was administered. Then, twenty-four hours after iv administration, the mice were sacrificed by an anesthetic, and then each tissue/organ was collected, and blood was collected by puncturing the inferior vena cava with a needle. After that, the intravascular lumens in each organ and tissue were washed by intermittently injecting 20 ml of a physiological saline containing 5 units/ml of heparin with a syringe to wash out the blood. Each about 100 mg of these tissue preparations was taken into a Falcon tube (15 ml). To the tube was added 0.25 ml of a 1:1 mixture of concentrated sulfuric acid and concentrated nitric acid, and then the sample was decomposed/digested at 80° C. for 2 hours. After cooling the sample, 10 ml of distilled water was added thereto and the mixture was well stirred with a Vortex mixer. Then, the sample was subjected to the measurement of boron content. Namely, 5 ml of this sample was taken in a new Falcon tube (10 ml), charged in ICP (Inductively coupled plasma)—mass spectrometry, and the amount of elements ¹⁰B and ¹¹B was quantified in unit of ppb. According to the results, it was found that ¹⁰B/¹¹B were almost equally well accumulated in the tumor sites (about 20 times higher than that of low molecular weight free boric acid) relative to normal organs. Therefore, it came to the conclusion that the macromolecular SGB as an anticancer drug was far superior to low molecular weight boric acid.

Based on the above results, the same experiment was performed. Mouse sarcoma S180 cells were inoculated (10⁶ cells) under the back skin of ddY mice. When the tumors were about 10-14 mm in diameter, free boric acid or SMA-glucosamine-boric acid complex (SGB) was dissolved in distilled water at 15 mg/kg, and about 0.1 ml of the solution was injected intravenously. After 24 hours of intravenous injection, the mice were slaughtered and blood, tumor tissue and other normal tissues (brain, lung, liver, spleen, kidney, etc.) were taken out. About 100 mg of each tissue sample was taken in a Falcon tube (15 ml) and 0.25 ml of a 1:1 solution of concentrated nitric acid and concentrated sulfuric acid was added thereto and the mixture was digested at 80° C. for 2 hours. The sample was cooled and 10 ml of distilled solution was added to each tube. Finally, ¹⁰B was quantified (ppb) by ICP MS (Agilent Technology, model 7900, Santa Clara, Calif., USA). The results are shown in FIG. 9. SGB was significantly more accumulated in the tumor sites than free boric acid.

### Test Example 7: Plasma Half-Lives and Urinary Excretion Rates of Boric Acid and SMA-Glucosamine-Boric Acid Complex in ddY Mice

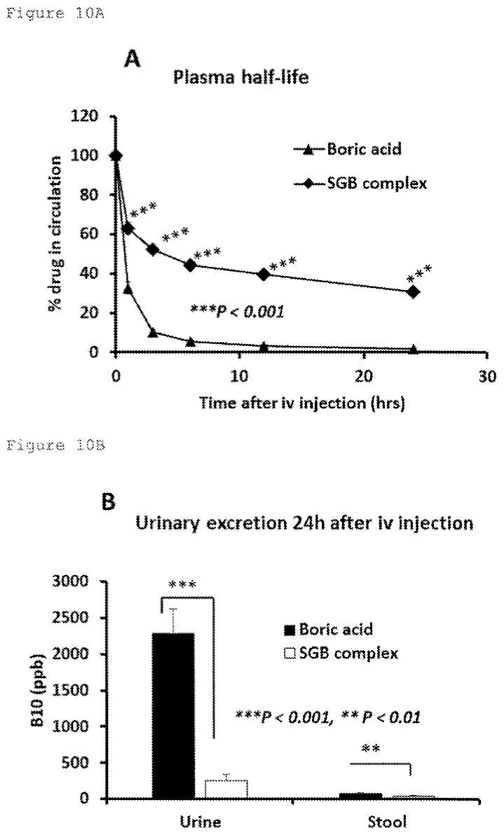

SGB and free boric acid were intravenously injected into ddY mice at a boric acid equivalent of 15 mg/kg each. After intravenous injection, blood samples were collected every 0, 3, 6, 12, and 24 hours and centrifuged to obtain plasma. The plasma was then treated in the same manner as in Test Example 6 above, and the blood concentration was calculated from the amount of boron in the plasma as the half-life of boric acid in the blood. The results are shown in FIG. 10A.

In addition, after 24 hours of intravenous injection, a thick filter paper (Whatman 3MM) was laid in the mouse cage and the adsorbed urine was collected on it, and the residual urine in the bladder was also collected with a syringe. Then, as in Test Example 6, the amount of boron was quantified by ICP MS and the urinary excretion rate was determined. The results are shown in FIG. 10B. SGB was excreted much less in urine than free boric acid.

### Test Example 8: Comparison of Cell Uptake of Free Boric Acid and SMA-Glucosamine-Boric Acid Complex (SGB) in C26 Cells

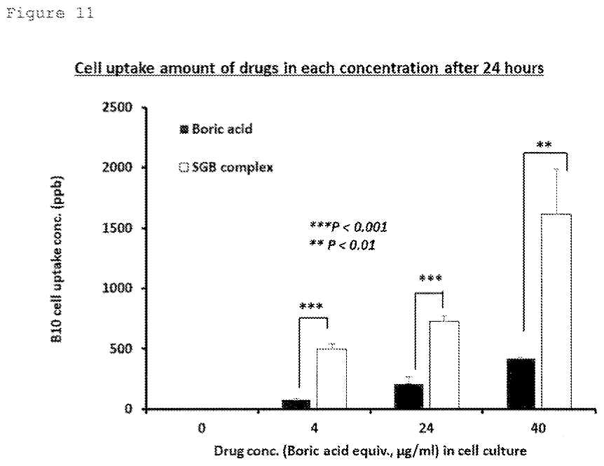

First, C26 cells (2×10⁴ cells/well) were cultured overnight in Eagle MEM medium containing 10% FBS in 24-well plates, then treated with boric acid or SGB, and then incubated at 37° C. Twenty-four hours after drug treatment, the cells were lysed by 0.1% Triton X 100 and the cell uptake amount of boron was measured by ICP MS. The results are shown in FIG. 11. The uptake of SGB borate was about 3-7 times higher than that of free boric acid.

### Test Example 9: Inhibition of Glucose Uptake and Lactate Production by SMA-Glucosamine-Boric Acid Complex

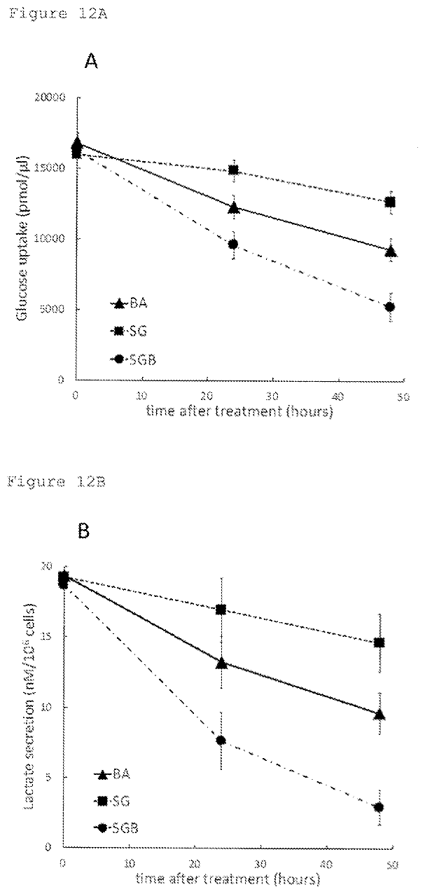

HeLa cells (1×10⁴ cells/well) were seeded in Falcon 96-well culture plates and cultured overnight in Eagle MEM under low oxygen partial pressure (pO₂ 1%). Cells were treated with boric acid (BA), SG (SMA-glucosamine), and SGB at a boric acid equivalent concentration of 100 μg/ml each. At predetermined times, glucose uptake (FIG. 12A) and lactate secretion (FIG. 12B) were measured according to the assay kit instructions from Dojindo Laboratories.

### Test Example 10: Enhancement of Antibacterial Activity of Boric Acid by SMA-Glucosamine-Boric Acid Complex (SGB)

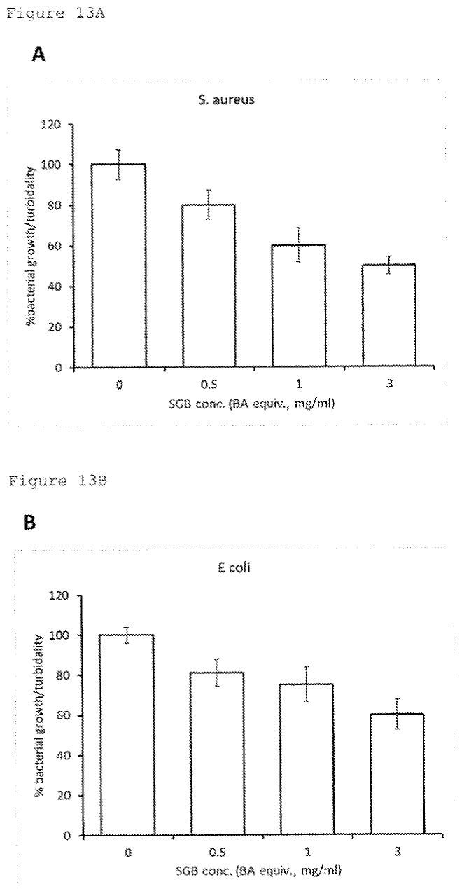

The antimicrobial activity of SGB was examined using the Gram-positive bacteria Staphylococcus aureus and Escherichia coli (E. coli) as pathogenic bacteria. The results are shown in FIGS. 13A and 13B.

Firstly, in a Falcon plastic plate with 96 wells, 0.1 ml of Mueller-Hinton medium was added to each well, followed by 10 μl of suspension of each bacterium (10⁴/well). SGB containing about 20% glucosamine and about 8% boric acid was used as the test sample. To each of these wells, 0, 0.5, 1.0, and 3 mg/ml of SGB (free boric acid equivalent) was added and incubated for 24 hours under constant temperature at 37° C. Twenty-four hours after the addition of SGB, the turbidity of this plate at 650 nm was measured and considered as an increase (inhibition) in the amount of bacteria.

Boric acid is used as an antibacterial substance in ophthalmology and other fields, where the concentration of boric acid is 10 mg/ml (1%) or higher. As shown in FIGS. 13A and 13B, SGB showed antibacterial activity against both Gram-positive bacteria (Staphylococcus aureus) and Gram-negative bacteria (E. coli) at concentrations of boric acid significantly lower than 10 mg/ml. In other words, it is clear that SGB shows stronger antibacterial activity than boric acid. The antibacterial activity of SGB was further increased when the same experiment was conducted under more anaerobic conditions.

1. A complex comprising a styrene-maleic acid copolymer (SMA) and a boric acid compound, wherein the SMA is bound to the boric acid compound directly or through a linker.

2. The complex according to claim 1, wherein the boric acid compound is selected from boric acid, disodium tetraborate, and mixtures thereof.

3. The complex according to claim 1, wherein the linker is bound to the SMA via an amide bond, an ester bond, a thioester bond, or a hydrazone bond.

4. The complex according to claim 1, wherein the linker is selected from saccharides, amino sugars, sugar alcohols, and mixtures thereof.

5. The complex according to claim 1, wherein the linker is a cis-diol compound.

6. The complex according to claim 1, wherein the SMA is directly bound to the boric acid compound.

7. An anticancer agent comprising the complex according to claim 1.

8. The anticancer agent according to claim 7 for use in boron neutron capture therapy.

9. A method for producing the complex according to claim 1, which comprises the following steps:
(a) binding the linker to the SMA, and
(b) binding the linker residue of the product obtained in step (a) to the boric acid compound.

10. An anticancer agent comprising a conjugate of styrene-maleic acid copolymer (SMA) and glucosamine.

11. An antibacterial agent comprising the complex according to claim 1.
